(19) United States
(12) Patent Application Publication
(10) Pub. No.: US 2014/0147926 A1
(43) Pub. Date: May 29, 2014

(54) METHOD OF ANALYZING ALDEHYDE COMPOUND IN METAL PLATING SOLUTION

(71) Applicant: SAMSUNG ELECTRO-MECHANICS CO., LTD., Suwon (KR)

(72) Inventors: Ji Eun Jeon, Suwon (KR); Se Kyung Lee, Suwon (KR); Hyo Jin Yoon, Suwon (KR); Suk Jin Ham, Suwon (KR)

(73) Assignee: SAMSUNG ELECTRO-MECHANICS CO., LTD., Suwon (KR)

(21) Appl. No.: 13/829,617

(22) Filed: Mar. 14, 2013

(30) Foreign Application Priority Data
Nov. 23, 2012 (KR) 10-2012-0133835

(51) Int. Cl. G01N 33/20

(52) U.S. Cl. CPC G01N 33/20
USPC 436/130; 436/128

(57) ABSTRACT

This invention relates to a method of analyzing an aldehyde compound in a metal plating solution, including adding a pH control solution to an aldehyde derivative, thus preparing an oversaturated aldehyde derivative solution in which the aldehyde derivative is dissolved to be oversaturated while the pH of the aldehyde derivative solution is adjusted to be the same as that of the metal plating solution; adding the oversaturated aldehyde derivative solution to the metal plating solution, so that the aldehyde compound which is present in the metal plating solution undergoes derivation, thus obtaining an aldehyde derivative compound; extracting the aldehyde derivative compound; and analyzing the aldehyde compound from the extracted aldehyde derivative compound.

### CROSS-REFERENCE TO RELATED APPLICATION

This application claims the benefit of Korean Patent Application No. 10-2012-0133835, filed Nov. 23, 2012, entitled “Analysis method for aldehyde compounds in metal plating solutions,” which is hereby incorporated by reference in its entirety into this application.

### BACKGROUND OF THE INVENTION

1. Technical Field

The present invention relates to a method of analyzing an aldehyde compound in a metal plating solution.

2. Description of the Related Art

Typically, aldehyde compounds such as formaldehyde have disinfecting actions and powerful reducing actions and thus are being widely used in a variety of fields. However, these compounds are known to be very toxic and to cause cancer in human beings and animals, and are defined as a carcinogen (Group 1) by the IARC (International Agency for Research on Cancer) and classified as a hazardous material which is carcinogenic and mutagenic by the EPA (Environmental Protection Agency) in the United States.

As environmentally hazardous problems associated with formaldehyde are a serious concern these days, the use of formaldehyde is strongly regulated in electric/electronic industries. In order to comply with such environmental regulations in the electric/electronic industries, it is necessary to establish methods for detection of formaldehyde in a plating solution and detection of a very small amount of formaldehyde for verification of an additive containing no formaldehyde.

General methods of measuring aldehyde compounds include colorimetric assay using acid or alkali titration, and gas chromatography (GC) or high performance liquid chromatography (HPLC) using chemical derivation.

For example, a method of analyzing an aldehyde component in air is performed in such a manner that a predetermined amount of air is sucked using a pump which operates by power so that an aldehyde compound is adsorbed on an adsorbent, after which pretreatment procedures and analysis operations are conducted in labs, followed by a process such as HPLC which requires expensive equipment which is complicated to operate, undesirably increasing analysis costs.

Also a method of detecting formaldehyde in an aqueous solution includes a series of complicated procedures of preparing a pH buffer solution adapted for a derivation reaction using a UV absorbing material, adding it, performing the derivation reaction using a UV absorbing material, performing filtration with a silica gel adsorbent and dewatering, and conducting 100% dewatering and re-extraction using an organic solvent, after which a UV absorption signal is analyzed using chromatography. For example, Patent Literature 1 discloses a method of quantitatively analyzing an aldehyde compound in soil, including subjecting an aldehyde compound to derivation, thus obtaining an aldehyde derivative compound, which is then quantitatively analyzed using HPLC.

Although such a method enables qualitative analysis of formaldehyde, it is limited in detecting formaldehyde having a concentration of 0.5 mg/ml or less. Furthermore, this method is problematic because pretreatment for extracting the derivative material using a pump that operates by power has to be carried out, as well as multiple analysis procedures, undesirably increasing analysis costs due to time and manpower requirements.

In addition, a variety of methods of detecting formaldehyde in air or a typical aqueous solution are known, but information about methods of detecting aldehyde compounds in metal plating solutions including metal composite materials such as metal ions, organic/inorganic acids, polymers, etc. has not yet been introduced. Moreover, the detection method which enables trace analysis at a concentration of 0.5 mg/ml or less is exemplified by chromatography, but methods are required that can be used to detect a ppb-level concentration of 0.1 mg/ml or less in order to achieve detection necessary for environmental regulations wherein the concentration of aldehyde is zero.

Patent Literature 1: Japanese Unexamined Patent Application Publication No. 2005-134274

### SUMMARY OF THE INVENTION

Culminating in the present invention, intensive and thorough research with the aim of solving the problems occurring in the related art led to the development of an aldehyde derivative solution and a method of analyzing an aldehyde compound using the same, wherein a very small amount of an aldehyde compound in a plating solution may be qualitatively and/or quantitatively detected in order to comply with environmental regulations in electric/electronic industries.

Accordingly, an aspect of the present invention is to provide a method of analyzing an aldehyde compound in a metal plating solution, in which an aldehyde compound in a metal plating solution may be simply and profitably detected up to a level of 0.1 mg/ml or less.

In order to accomplish the above aspect, the present invention provides a method of analyzing an aldehyde compound in a metal plating solution, comprising adding a pH control solution to an aldehyde derivative, thus preparing an oversaturated aldehyde derivative solution in which the aldehyde derivative is dissolved to be oversaturated while the pH of the aldehyde derivative solution is adjusted to be the same as that of the metal plating solution; adding the oversaturated aldehyde derivative solution to the metal plating solution, so that the aldehyde compound which is present in the metal plating solution undergoes derivation, thus obtaining an aldehyde derivative compound; extracting the aldehyde derivative compound; and analyzing the aldehyde compound from the extracted aldehyde derivative compound.

In the method of the invention, the aldehyde derivative may be one or more selected from the group consisting of acetylacetone, oxazolidine, o-(pentafluorobenzyl)-hydroxylamine, 2,4-dinitrophenylhydrazine, 2,3,4,5,6-pentafluorophenylhydrazine, 2-aminoethanethiol, and 2,4,6-trichlorophenylhydrazine.

In the method of the invention, the pH control solution may be an inorganic acid solution comprising hydrochloric acid, sulfuric acid, nitric acid, phosphoric acid or a mixture thereof, or may be an alkaline solution comprising sodium hydroxide, potassium hydroxide, sodium carbonate or a mixture thereof.

In the method of the invention, the pH of the oversaturated aldehyde derivative solution may be set in the range of metal plating solution pH±2.

In the method of the invention, the oversaturated aldehyde derivative solution may be prepared via heating in the temperature range of 50˜80° C. for a period of time ranging from 20 min to 1 hr.

In the method of the invention, the oversaturated aldehyde derivative solution and the metal plating solution may be mixed at a volume ratio of 1:100˜200.

In the method of the invention, the plating solution may be an electrolytic or electroless plating solution comprising a metal ion, an inorganic acid, an organic polymer and an organic monomer, which are mixed together.

In the method of the invention, the aldehyde compound may be one or more selected from the group consisting of formaldehyde, acetaldehyde, acrolein, acetone, propionaldehyde, crotonaldehyde, butyraldehyde, benzaldehyde, i-valeraldehyde, n-valeraldehyde, o-valeraldehyde, m-valeraldehyde, p-valeraldehyde, hexaldehyde and 2,5-dimethylbenzaldehyde.

In the method of the invention, extracting the aldehyde derivative compound may be performed by dissolving the aldehyde derivative compound in an organic solvent, removing the organic solvent, and then performing drying and extraction.

In the method of the invention, the organic solvent may be one or more selected from the group consisting of methylene chloride, chloroform, n-hexane, diethyl ether, ethyl acetate, and carbon tetrachloride.

In the method of the invention, analyzing the aldehyde compound may be performed via either or both of quantitative analysis and qualitative analysis.

In the method of the invention, analyzing the aldehyde compound may be performed using HPLC-MS/MS (High Performance Liquid Chromatography-Mass/Mass Spectrometry).

The method of the invention enables detection of an aldehyde compound having a concentration of 0.1 mg/l or less.

### BRIEF DESCRIPTION OF THE DRAWINGS

The above and other aspects, features and other advantages of the present invention will be more clearly understood from the following detailed description when taken in conjunction with the accompanying drawings, in which:

FIG. 1 is a block diagram illustrating a process of analyzing an aldehyde derivative compound in a metal plating solution according to an embodiment of the present invention;

FIG. 2 illustrates spectrums of an aldehyde derivative compound at different concentrations, as analyzed using HPLC-MS/MS (High Performance Liquid Chromatography-Mass/Mass Spectrometry) using a standard reagent to perform quantitative analysis of an aldehyde derivative compound, according to the present invention;

FIG. 3 is a graph illustrating a standard calibration curve depending on the concentration using the spectrums of the aldehyde derivative compound at different concentrations of FIG. 2;

FIG. 4 is a spectrum illustrating the results of analysis of the aldehyde derivative compound via HPLC-MS/MS according to an embodiment of the present invention;

FIG. 5 illustrates spectrums for the signal of the aldehyde derivative compound detected using both a UV detector and a MS detector according to an embodiment of the present invention;

FIG. 6 illustrates spectrums of the aldehyde derivative compound obtained by analyzing the aldehyde derivative compound three times using HPLC-MS/MS according to an embodiment of the present invention; and

FIG. 7 is a graph illustrating the results of quantitative analysis of the aldehyde compound in a plating solution, obtained by substituting the spectrums of the aldehyde derivative compound analyzed three times of FIG. 6 into the calibration curve of FIG. 3.

### DESCRIPTION OF SPECIFIC EMBODIMENTS

Before the present invention is described in more detail, the terms and words used in the present specification and claims should not be interpreted as being limited to typical meanings or dictionary definitions, but should be interpreted as having meanings and concepts relevant to the technical scope of the present invention based on the rule according to which an inventor can appropriately define the concept implied by the term to best describe the method he or she knows for carrying out the invention. It is noted that, the embodiments of the present invention are merely illustrative, and are not construed to limit the scope of the present invention, and thus there may be a variety of equivalents and modifications able to substitute for them at the point of time of the present application.

In the following description, it is to be noted that embodiments of the present invention are described in detail so that the present invention may be easily performed by those skilled in the art, and also that, when known techniques related with the present invention may make the gist of the present invention unclear, a detailed description thereof will be omitted.

### DEFINITION OF TERMS

The terms used in the present invention are defined as follows.

In the present invention, the term “aldehyde derivative” means a material which forms an aldehyde derivative compound via reaction with an aldehyde compound which is present in a metal plating solution. The term “aldehyde compound” means an aldehyde series or aldehyde-based material which is defined as a carcinogen (Group 1) by the IARC and is classified as a hazardous material which is carcinogenic and mutagenic by the EPA. The term “metal plating solution” indicates an electrolytic/electroless plating solution containing a metal such as copper which is mainly used in a printed circuit board, but the present invention is not necessarily limited thereto. The term “aldehyde derivative solution” means a mixture of an aldehyde derivative and a pH control solution. The term “aldehyde derivative compound” means a reaction product obtained by reacting the aldehyde derivative with the aldehyde compound that is present in the metal plating solution.

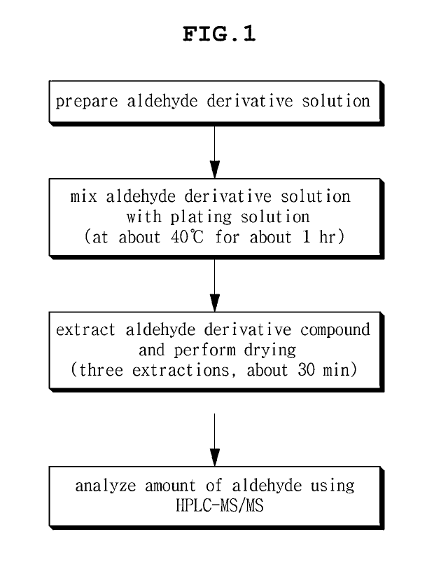

FIG. 1 illustrates a process of analyzing an aldehyde derivative compound in a metal plating solution according to an embodiment of the present invention.

Typically, a metal plating solution which is in an aqueous solution phase cannot be subjected to an extraction method in air. A method of analyzing formaldehyde in an aqueous solution may include a colorimetric assay method using acid/alkali titration, such as an ammonium chloride method, a hydroxylamine hydrochloride method, etc. In the case of a titration method, it is a wet assay method in which detection error may increase depending on raters, measurement equipment and environment, making it difficult to utilize in quantitative analysis of a very small amount of a sample at 1 ng/l or less. Also, in the course of titration, neutralization of the plating solution in a state of strong acid/strong base may cause precipitation and generation of heat, undesirably losing an additive, etc. Hence, the above method cannot be applied to the plating solution in an aqueous solution phase.

In addition, a variety of methods of detecting formaldehyde in air or a typical aqueous solution are known, but information about detection of formaldehyde in a metal plating solution including metal composite materials such as a metal ion, an inorganic acid, and/or a polymer is not specifically known. In order to detect formaldehyde having a concentration of about 0.5 mg/l in a metal plating solution, chromatography may be used. However, this method is difficult to apply to the detection necessary for environmental regulations wherein the concentration of aldehyde is zero or is a ppb level of 0.1 mg/l or less. This is because, in the case of a plating solution containing an excess of metal ion, generation of heat and precipitation may occur due to a neutralization reaction when applying a buffer solution for use in chromatography. Thus, the above method makes it difficult to serve in quantitative assay, and furthermore to serve in detecting a ppb-level concentration of 0.1 mg/l or less.

As mentioned above, formaldehyde in the electrolytic/electroless plating solution may be mainly used as a reducing agent which reduces a metal ion such as silver or copper to conduct plating (metal growth). This component is utilized as a plating additive, such as an anti-corrosive agent, an acid inhibitor, a catalyst, etc., and in particular, has to be essentially added to the electroless plating solution. Formaldehyde which has a great influence on plating characteristics may affect a reaction rate, plating solution stability, and surface roughness depending on the added amount thereof, and thus it is important to control the concentration of formaldehyde in the plating solution.

In the present invention, an improved chemical derivative pretreatment method is used, so that an aldehyde compound in the plating solution containing an excess of metal ion is extracted with an organic solvent without complicated pretreatment procedures including silica gel filtration, vacuum pumping, buffer solution addition, dewatering and so on. The extracted aldehyde derivative compound may be subjected to analysis of a very small amount of formaldehyde (quantitative analysis), as well as qualitative analysis using a variety of tandem MS methods including HPLC-MS/MS.

The present invention provides a method of detecting the amount of aldehyde by subjecting an aldehyde component which is present in a plating solution including a metal ion, an inorganic acid and/or an organic acid, an organic polymer, an organic monomer, and/or other additives to a series of processes of derivation, extraction with an organic solvent, and HPLC-MS/MS which enables qualitative/quantitative analysis of a small amount of an organic material at 0.1 mg/ml or less.

Also, the present invention provides an analysis method which may be utilized in a pretreatment method in which an aldehyde compound in a plating solution is subjected to derivation using an aldehyde derivative and in a method of measuring a very small amount of aldehyde at 0.1 mg/l or less necessary to verify an environmentally regulated material in future.

According to the present invention, in order to measure the amount of an aldehyde compound at 0.1 mg/l or less in a plating solution, pretreatment of an aldehyde derivative is first performed. Upon pretreating the aldehyde derivative according to the present invention, it is important to perform derivation of the aldehyde compound without interference from a metal ion and without generation of heat and precipitation due to a neutralization reaction during derivation of the aldehyde compound in the metal plating solution.

FIG. 1 illustrates a process of analyzing an aldehyde derivative compound in a metal plating solution according to an embodiment of the present invention.

With reference to FIG. 1, a pH control solution is added to an aldehyde derivative, thus preparing an oversaturated aldehyde derivative solution in which the aldehyde derivative is dissolved to be oversaturated while the pH of the aldehyde derivative solution is adjusted to be substantially the same as that of the metal plating solution. In this case, the pH of the aldehyde derivative solution is adjusted within the range of the metal plating solution pH±2.

As the pH of the aldehyde derivative solution thus prepared is maintained to be substantially the same as that of the plating solution, generation of heat, precipitation and/or decomposition may be prevented from occurring due to a neutralization reaction, and the plating solution may remain undiluted. In the case where the generation of heat, precipitation and/or decomposition take place, the aldehyde compound may be captured by the generated precipitate, making it difficult to perform accurate quantitative analysis. If the pH of the oversaturated aldehyde derivative solution falls out of the range of the metal plating solution pH±2, generation of heat and precipitation may occur, undesirably producing a precipitate.

The aldehyde derivative usable in the present invention may be one or more selected from among materials which form hydrazone, thiazolidine and oxime derivatives, including acetylacetone, oxazolidine, o-(pentafluorobenzyl)-hydroxylamine (PFBHA), 2,4-dinitrophenylhydrazine (2,4-DNPH), 2,3,4,5,6-pentafluorophenylhydrazine (2,3,4,5,6-PFPH), 2-aminoethanethiol(cysteamine), 2,4,6-trichlorophenylhydrazine (TCPH), etc.

In the case where the metal plating solution is acidic, the pH control solution may be an inorganic acid solution including hydrochloric acid, sulfuric acid, nitric acid, phosphoric acid or a mixture thereof. When the metal plating solution is basic, an alkaline solution including sodium hydroxide, potassium hydroxide, sodium carbonate or a mixture thereof may be used.

Also according to the present invention, in order to measure the amount of the aldehyde compound at 0.1 mg/l or less in a metal plating solution, a sufficient amount of the aldehyde derivative should be added to the plating solution. Thus, the aldehyde derivative solution the pH of which was adjusted by the pH control solution is heated in the temperature range of 50˜80° C. for a period of time ranging from 20 min to 1 hr, thus preparing an oversaturated aldehyde derivative solution. If the temperature is lower than 50° C., the extent of saturation of the aldehyde derivative may decrease. In contrast, if the temperature is higher than 80° C., the amount of the pH control solution to be volatilized may increase, undesirably changing the pH. For the same reason, the reaction time may be set to the range of 20 min to 1 hr.

Subsequently, the oversaturated aldehyde derivative solution is added to the metal plating solution so that the aldehyde compound which is present in the metal plating solution undergoes derivation, thus obtaining an aldehyde derivative compound.

In the case where the aldehyde compound is formaldehyde and the aldehyde derivative is 2,4-DNPH, a derivation reaction of the aldehyde compound is represented by Scheme 1 below.

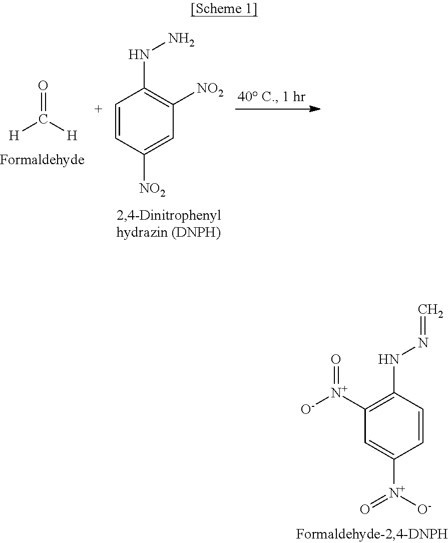

In the present invention, the mixing ratio of the oversaturated aldehyde derivative solution to the metal plating solution is 1:100˜200 by volume. If the mixing ratio is less than 100, the reaction between the aldehyde derivative and the aldehyde compound does not sufficiently occur. In contrast, if the mixing ratio exceeds 200, there is no profitability.

The aldehyde compound which may be analyzed according to the present invention may be one or more selected from the group consisting of formaldehyde, acetaldehyde, acrolein, acetone, propionaldehyde, crotonaldehyde, butyraldehyde, benzaldehyde, valeraldehyde, n-valeraldehyde, o-valeraldehyde, m-valeraldehyde, p-valeraldehyde, hexaldehyde and 2,5-dimethylbenzaldehyde.

According to the present invention, the aldehyde derivative compound thus obtained is dissolved in an organic solvent, after which the separated organic solvent layer is recovered. The organic solvent is removed via volatilization using a typical method from the organic solvent layer, followed by performing drying in an oven or the like, thus obtaining a solid aldehyde derivative compound.

In the present invention, any organic solvent may be selectively used so long as it has low solubility in water, high polarity, and high volatility to shorten the drying time. The aldehyde derivative compound is extracted with the organic solvent, and then stored after removal of the organic solvent. The organic solvent useful in the present invention may be one or more selected from the group consisting of methylene chloride, chloroform, n-hexane, diethyl ether, ethyl acetate, and carbon tetrachloride.

Subsequently, the extracted aldehyde derivative compound may be subjected to either or both of quantitative analysis and qualitative analysis using a variety of methods. When using HPLC-MS/MS, it is possible to detect an aldehyde compound having a concentration of 0.1 mg/l or less. The HPLC-MS/MS method is high-resolution mass spectrometry able to detect not only high-concentration aldehyde but also a very small amount of aldehyde. In particular, the HPLC-MS/MS method performs double mass spectrometry of a target compound via purification, thereby very accurately measuring the mass of the target compound. The HPLC-MS/MS method has a detection limit of ppb-level concentration of 0.5 mg/l or less, compared to typical chromatography (GC or HPLC).

A better understanding of the present invention may be obtained via the following examples which are set forth to illustrate, but are not to be construed as limiting the present invention.

### Preparation Example 1

### Preparation of Electrolytic Copper Sulfate Plating Solution Sample

1 l of pure water was mixed with 140 g of sulfuric acid (H₂SO₄, 60%), 30 g of copper sulfate (CuSO₄), and 30 ppm of hydrochloric acid (HCl, 35%), thus preparing a plating solution having a pH of about 2.5, after which 25 mg of polyethylene glycol (PET 4000) having 4000 Da was finally added, thus preparing an electrolytic copper sulfate plating solution.

### Preparation Example 2

### Preparation of Formaldehyde Derivative Solution

As a formaldehyde derivative, 2,4-DNPH and 100 ml of a hydrochloric acid solution were mixed at about 70° C. for about 30 min, thus preparing a 0.01M 2,4-DNPH solution (pH of about 2.5).

### Preparation Example 3

### Formation of Calibration Curve Depending on Concentration

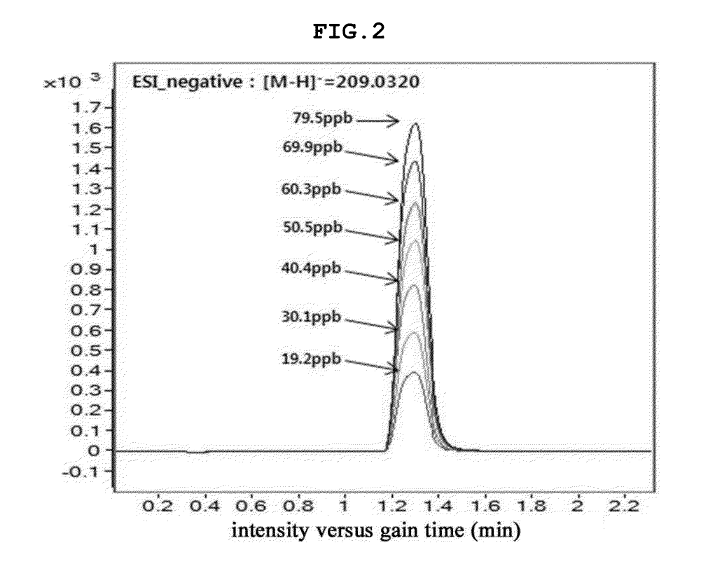

In order to accurately analyze the concentration of formaldehyde in the metal plating solution of Preparation Example 1 (quantitative analysis), formaldehyde was added at different concentrations of 10˜80 ppb (ng/l) to the plating solution. Subsequently, 1 ml of this mixture was mixed with 150 ml of the 0.01M 2,4-DNPH solution (pH of about 2.5) of Preparation Example 2 so that they reacted at about 40° C. for about 1 hr, thus synthesizing formaldehyde-2,4-DNPH. Subsequently, this reaction product was mixed with methylene chloride at a volume ratio of 1:1, so that the formaldehyde-2,4-DNPH was extracted with an organic layer, the organic layer was separated, and the methylene chloride was volatilized, thus obtaining a dried solid formaldehyde-2,4-DNPH. This compound was placed in a HPLC-MS/MS analyzer, and mass spectrums thereof were measured at different concentrations. The results are shown in FIG. 2.

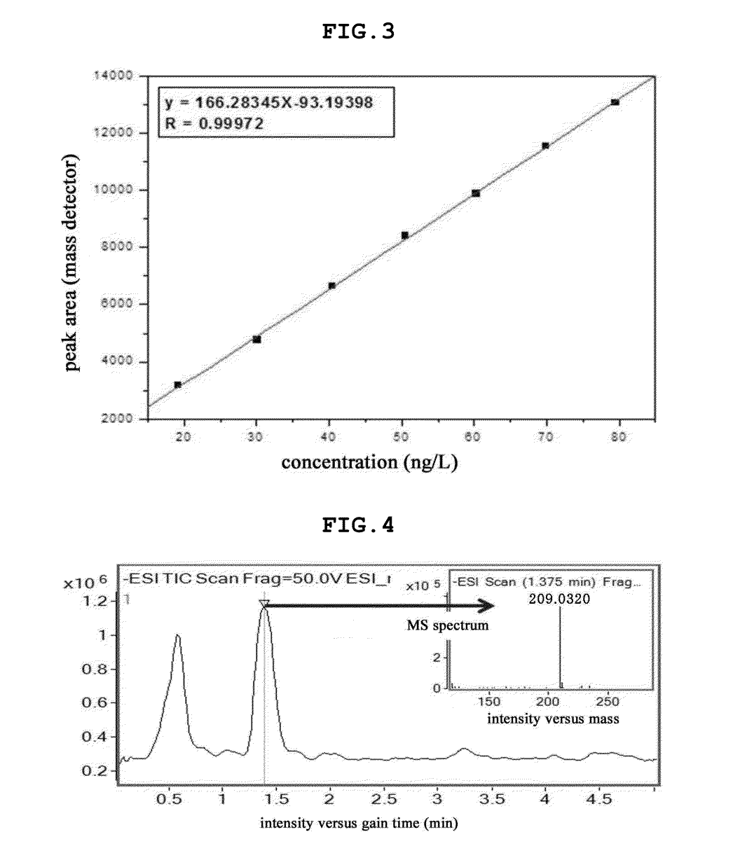

On the other hand, a formaldehyde-2,4-DNPH solution having a formaldehyde concentration of 10˜80 ppb (ng/l) was placed in a HPLC-MS/MS analyzer, and the mass spectrums thereof were measured at different concentrations. These results were substantially the same as those of FIG. 2. Thus, using the HPLC-MS spectrums of FIG. 2, a standard calibration curve depending on the concentration is shown in FIG. 3. The concentration range of the calibration curve is 10˜80 ppb (ng/l), and linearity depending on the concentration is good (gradient (R)=0.9997).

### Example 1

1 ml of the metal plating solution of Preparation Example 1 containing formaldehyde the amount of which was unknown was mixed with 150 ml of the 0.01M 2,4-DNPH solution (pH of about 2.5) of Preparation Example 2 so that they reacted at about 40° C. for about 1 hr, thus synthesizing formaldehyde-2,4-DNPH. Subsequently, this reaction product was mixed with methylene chloride at a volume ratio of 1:1, so that the formaldehyde-2,4-DNPH was extracted with an organic layer, the organic layer was separated, and the methylene chloride was volatilized, thus obtaining a dried solid formaldehyde-2,4-DNPH. This product was placed in a HPLC-MS/MS analyzer, the mass spectrum of which was measured. The results are shown in FIG. 4. As illustrated in FIG. 4, the peak of formaldehyde-2,4-DNPH having a molecular weight of 209.0320 can be observed.

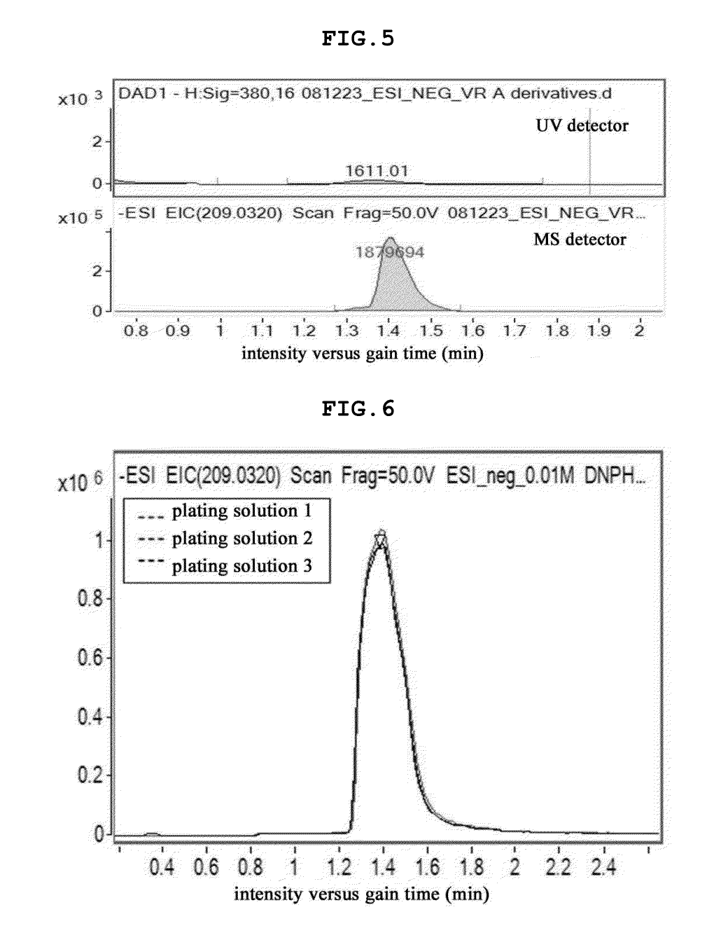

Furthermore, FIG. 5 illustrates spectrums for the signal of the formaldehyde-2,4-DNPH detected using both a UV detector and a MS detector. The area of the spectrum detected using the UV detector was 1611.01, whereas the area of the spectrum detected using the MS detector was 1879694. As such, there is a difference of at least 1100 times therebetween. Also, although absorption takes place at a wavelength range of 350˜500 nm when using the UV detector, the case wherein a very small amount of aldehyde is contained is limited in detecting the absorption signal in ppb level even when the UV detector having very high sensitivity is used.

### Example 2

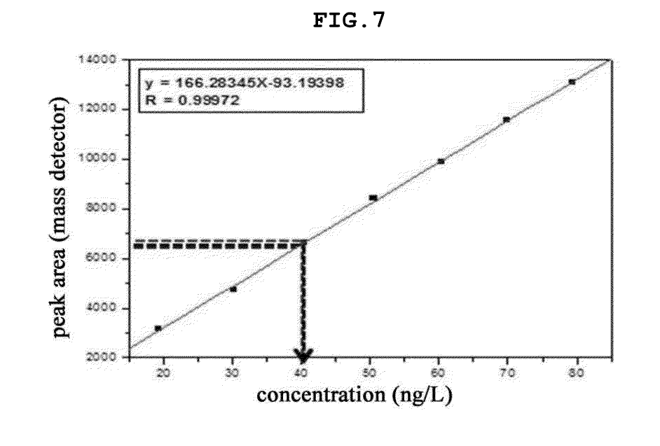

A solid formaldehyde-2,4-DNPH obtained in the same manner as in Example 1 was placed in a HPLC-MS/MS analyzer, and mass spectrum thereof was measured three times. The results are shown in FIG. 6. The peak area obtained from the above spectrum was substituted into the standard calibration curve depending on the concentration of FIG. 3 obtained in Preparation Example 3, and thereby the amount of aldehyde in the metal plating solution was determined to be about 40 ng/l (FIG. 7).

As described hereinbefore, the present invention provides a method of analyzing an aldehyde compound in a metal plating solution. According to the present invention, the aldehyde compound can be simply and profitably detected up to a concentration of 0.1 mg/l or less.

Although the embodiments of the present invention have been disclosed for illustrative purposes, those skilled in the art will appreciate that a variety of different modifications, additions and substitutions are possible, without departing from the scope and spirit of the invention as disclosed in the accompanying claims.

Accordingly, such modifications, additions and substitutions should also be understood as falling within the scope of the present invention.

What is claimed is:

1. A method of analyzing an aldehyde compound in a metal plating solution, comprising:
adding a pH control solution to an aldehyde derivative, thus preparing an oversaturated aldehyde derivative solution in which the aldehyde derivative is dissolved to be oversaturated while a pH of the aldehyde derivative solution is adjusted to be same as that of the metal plating solution;
adding the oversaturated aldehyde derivative solution to the metal plating solution, so that the aldehyde compound which is present in the metal plating solution undergoes derivation, thus obtaining an aldehyde derivative compound;
extracting the aldehyde derivative compound; and
analyzing the aldehyde compound from the extracted aldehyde derivative compound.

2. The method of claim 1, wherein the aldehyde derivative is one or more selected from the group consisting of acetylacetone, oxazolidine, o-(pentafluorobenzyl)-hydroxylamine, 2,4-dinitrophenylhydrazine, 2,3,4,5,6-pentafluorophenylhydrazine, 2-aminoethanethiol, and 2,4,6-trichlorophenylhydrazine.

3. The method of claim 1, wherein the pH control solution is an inorganic acid solution comprising hydrochloric acid, sulfuric acid, nitric acid, phosphoric acid or a mixture thereof, or is an alkaline solution comprising sodium hydroxide, potassium hydroxide, sodium carbonate or a mixture thereof.

4. The method of claim 1, wherein the pH of the oversaturated aldehyde derivative solution is set in a range of metal plating solution pH±2.

5. The method of claim 1, wherein the oversaturated aldehyde derivative solution is prepared via heating in a temperature range of 50˜80° C. for a period of time ranging from 20 min to 1 hr.

6. The method of claim 1, wherein the oversaturated aldehyde derivative solution and the metal plating solution are mixed at a volume ratio of 1:100˜200.

7. The method of claim 1, wherein the plating solution is an electrolytic or electroless plating solution comprising a metal ion, an inorganic acid, an organic polymer and an organic monomer, which are mixed together.

8. The method of claim 1, wherein the aldehyde compound is one or more selected from the group consisting of formaldehyde, acetaldehyde, acrolein, acetone, propionaldehyde, crotonaldehyde, butyraldehyde, benzaldehyde, i-valeraldehyde, n-valeraldehyde, o-valeraldehyde, m-valeraldehyde, p-valeraldehyde, hexaldehyde and 2,5-dimethylbenzaldehyde.

9. The method of claim 1, wherein the extracting the aldehyde derivative compound is performed by dissolving the aldehyde derivative compound in an organic solvent, removing the organic solvent, and then performing drying and extraction.

10. The method of claim 9, wherein the organic solvent is one or more selected from the group consisting of methylene chloride, chloroform, n-hexane, diethyl ether, ethyl acetate, and carbon tetrachloride.

11. The method of claim 1, wherein the analyzing the aldehyde compound is performed via either or both of quantitative analysis and qualitative analysis.

12. The method of claim 1, wherein the analyzing the aldehyde compound is performed using HPLC-MS/MS (High Performance Liquid Chromatography-Mass/Mass Spectrometry).

13. The method of claim 1, which enables detection of an aldehyde compound having a concentration of 0.1 mg/l or less.
